(19) United States
(12) Patent Application Publication
(10) Pub. No.: US 2011/0033933 A1
(43) Pub. Date: Feb. 10, 2011

(54) METHOD APPLYING HEMODYNAMIC FORCING AND KLF2 TO INITIATE THE GROWTH AND DEVELOPMENT OF CARDIAC VALVES

(76) Inventors: Morteza Gharib, San Marino, CA (US); Derek Rinderknecht, Arcadia, CA (US); Arian Forouhar, Pasadena, CA (US); Julien Vermot, Strasbenrg (FR); Scott E. Fraser, La Canada, CA (US)

Correspondence Address: BERLINER & ASSOCIATES, 555 WEST FIFTH STREET, 31ST FLOOR, LOS ANGELES, CA, 90013, US

(21) Appl. No.: 12/837,369

(22) Filed: Jul. 15, 2010

Related U.S. Application Data
(60) Provisional application No. 61/225,646, filed on Jul. 15, 2009.

(51) Int. Cl. C12N 5/071; C12M 3/00

(52) U.S. Cl. 435/395; 435/325; 435/289.1

(57) ABSTRACT

A method for forming a cardiovascular structure in culture is provided. The method includes applying mechanical force to a cell population in culture such that a cardiovascular structure is formed. In some embodiments, the mechanical force is produced in culture medium by a pulsatile liquid flow with a retrograde component. The cell population can include stem cells or differentiated cells, or combinations of both. In particular embodiments, a cardiovascular valve is formed. Scaffolds for the support and growth of the cell population, and bioreactors including the scaffolds, are also provided.

### CROSS-REFERENCE TO RELATED APPLICATIONS

This application claims the benefit of Provisional Patent Application No. 61/225,646, filed on Jul. 15, 2009, which is incorporated by reference herein.

### BACKGROUND

1. Field of the Invention

This invention relates to an apparatus and method for tissue engineering and cardiovascular valve development.

2. Related Art

Valvulogenesis is a complex developmental event critical to the proper functioning of the cardiovascular system. Congenital heart defects affect 1 in every 100 live births. Obstruction defects in the cardiac and venous valves is one of the most common subcategories of these abnormalities, accounting for greater than 25% of all cardiac valve cases. Although mechanical (or prosthetic) valves made of synthetic materials can tolerate the physical stresses applied by the circulatory system, and are widely used in the clinic, their use requires lifetime anticoagulation therapy. Thus, other ways to treat valve defects are desirable.

The process of valvular development involves the tightly regulated expression of several growth factors and their receptors. Previous studies of embryonic zebrafish valvulogenesis have identified a key valvular morphologic event as the activation of a shear responsive gene upregulating the expression of Klf2 (Hove, J. R., et al., (2003) Nature 421, 172-177; Vermot, J., et al., (2009) PLoS Biol 7(11): e1000246.

doi:10.1371/journal.pbio.1000246).

### SUMMARY

Certain features of embryonic cardiovascular valve formation are applied to stimulate cardiovascular valve growth in vitro and in vivo. In particular, in some embodiments, mechanical forcing and Klf2 are used to orchestrate the in vivo and in vitro development of cardiovascular valves. Such embodiments provide a powerful therapeutic approach for regulating heart valve development and morphology, and can be used to prepare valves and other cardiovascular structures for transplantation into subjects. In addition, because Klf2 expression is important for the integrity of other vascular structures such as arteries and veins, certain embodiments of these methods can be applied to form active cardiac assist devices, promote vasculogenesis or angiogenesis, and influence endothelial or stem cell differentiation. Particular embodiments can also be used to create biological microfluidic components on a chip to replace standard synthetic components in current lab-on-chip technologies.

In one aspect, a method of forming a cardiovascular structure in culture is provided. The method includes applying mechanical force to a cell population in culture such that a cardiovascular structure is formed. In various embodiments, the mechanical force comprises a shear force, is a result of pulsatile retrograde fluid flow, or is transferred through a fluid or cell culture medium, or any combination thereof. In particular embodiments, the mechanical force, acting as an epigenetic factor for cardiovascular development, corresponds to a negative shear force resulting from retrograde flow greater than −0.01 dyn/cm².

For any embodiment, the cell population can include multipotent, pluripotent or totipotent cells, or cardiovascular cells, or a combination thereof. In addition, the formed cardiovascular structure can be a blood vessel or a valve. Further, the cell population can be supported by a scaffold prepared from an explanted cardiovascular structure. Further still, the mechanical force can be produced by forming a constriction that creates a local increase in the magnitude of retrograde shear force on the cell population.

The method can further include increasing expression of an endogenous or inserted Klf2 gene, Notch gene, BMP gene, or a combination thereof in the cell population. The increased expression can occur before, during or after applying the mechanical force, or a combination of thereof.

In another aspect, a method of forming a cardiovascular valve in culture is provided. The method includes applying retrograde fluid flow to a cell population in culture such that a cardiovascular valve is formed. In some embodiments, the retrograde fluid flow is pulsatile retrograde fluid flow. In some embodiments, the retrograde fluid flow is applied through a cell culture medium In any embodiment, the cell population can include multipotent, pluripotent or totipotent cells, or cardiovascular cells, or a combination thereof. Further, the cell population can be supported by a scaffold prepared from an explanted vein or artery.

The method can further include increasing expression of an endogenous or inserted Klf2 gene, Notch gene, or BMP gene, or a combination thereof, in the cell population wherein the increasing expression occurs before, during or after applying the retrograde fluid flow, or a combination of thereof.

In an additional aspect, a culture vessel that applies a mechanical force to a cell population is provided. The culture vessel includes a scaffold for supporting a cell population and means for producing a pulsatile fluid flow in the culture vessel. The scaffold includes at least one section where retrograde fluid flow results from movement of the pulsatile fluid flow through the scaffold. The scaffold can be a synthetically prepared support or a component of a cardiovascular system, or a combination of both. In some embodiments, the culture vessel is a bioreactor or a flow cell.

In a further aspect, a valve or other cardiovascular structure, prepared by an embodiment of the invention, is provided.

### BRIEF DESCRIPTION OF THE DRAWINGS

For a more complete understanding of the present invention, reference is now made to the following descriptions taken in conjunction with the accompanying drawing, in which:

FIGS. 1A and 1B are schematic representations illustrating pulsatile fluid flow with a retrograde component;

FIGS. 2A-C are schematic representations illustrating the production of retrograde fluid flow; and

FIG. 3 is a drawing of a bioreactor system.

### DETAILED DESCRIPTION

Although not wishing to be bound by any theory, the general view of the inventors is that certain features of embryonic valve development can be applied to produce cardiovascular valves and other structures in vivo and in vitro. Accordingly, in various embodiments, mechanical forces and Klf2 expression, which have been shown to be involved in embryonic valve development, are used for tissue engineering of cardiovascular structures.

A cardiovascular structure includes, but is not limited to, a cardiovascular valve including bileaflet or trileaflet valves, or a blood vessel including an artery, vein, capillary, arteriole, or venule. A cardiovascular valve can be a cardiac valve, venous valve, bileaflet valve, trileaflet valve, or other valve of the cardiovascular system. A cardiovascular valve can be defined as a biological device (i.e., containing cells or tissues) that regulates flow through a passage by being open, partially open or closed. In various embodiments, a valve can be functionally identified as a tissue capable of rectifying or blocking fluid flow. Morphologically, valve formation can be identified as the beginning of out-of-plane growth of a region of the cell population.

In some embodiments of the invention, a mechanical force is applied to a cell population in culture to form a cardiovascular structure. The mechanical force can be a fluid shear force, or any pressure that imposes tangential or radial stresses on the surface of the cell culture. In particular embodiments, mechanical forces are generated by producing retrograde flow in a fluid in contact with the cell population. For example, the fluid can be a culture medium, a physiological salt solution, or any combination thereof.

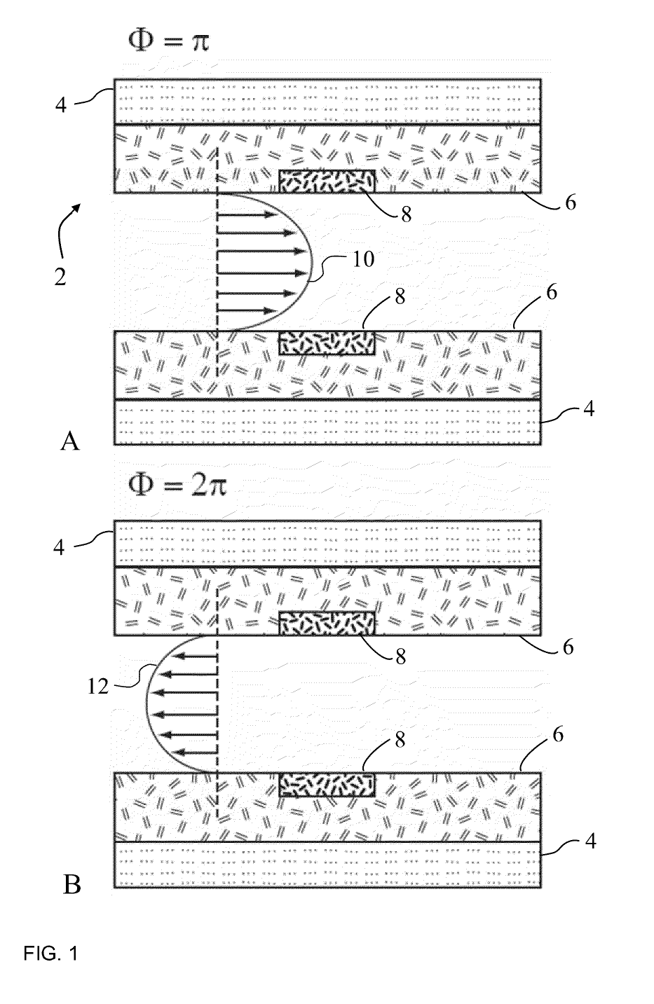

Retrograde fluid flow refers to the reversal in flow direction of a fluid, where the reversal varies either periodically or aperiodically in time. The reversal in fluid flow direction imposes a retrograde (or negative) shear force on the cell population. Retrograde flow can be produced using a variety of pumping systems, including positive-displacement systems such as, but not limited to, a piston pump, a peristaltic pump, a rotary vane pump, or a syringe pump, or a combination thereof. Also, retrograde flow can be produced using a system such as, but not limited to, an oscillating pressure system, a reciprocating mechanism system capable of creating oscillatory flow, such as a diaphragm-containing mechanism, or a push/pull syringe pump system capable of creating pressure driven flow. For example, referring to FIG. 1 which illustrates a way of creating retrograde flow, a portion of a bioreactor 2 is shown in cross section. The bioreactor includes a wall 4 and a scaffold 6 containing a cell population 8. At a particular point it in the phase cycle, the liquid flow 10 is in the forward direction, while at the following point 2 it in the phase cycle, the liquid flow 12 is in the reverse direction. The magnitude of flow 10 in the forward direction is greater than the magnitude of flow 12 in the reverse direction such that the overall flow is a pulsatile flow in the forward direction with a reverse flow component. As is apparent, the particular pulsatile flow and reverse flow component will depend on the flow rate of flows 10 and 12 and their frequency of reversal. In some cases, the reversal in flow direction can create time-dependent flow separation.

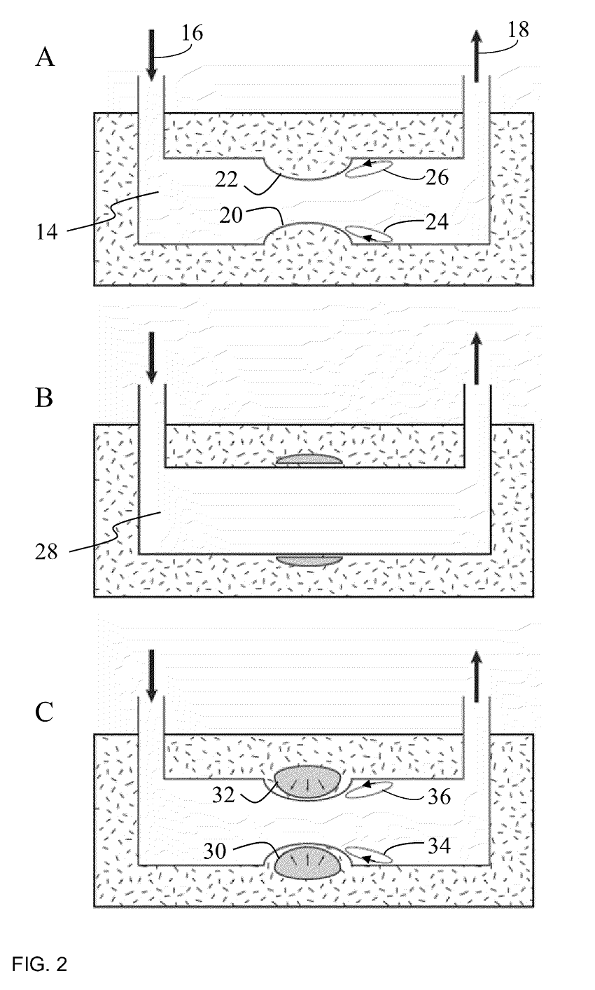

Retrograde flow can be produced at a particular location by creating a region of increased shear stress, for example, through a constriction or other mechanism that alters the cross sectional flow area of a vessel or conduit. Thus, valve formation can be arranged to occur at particular locations in a cell population, and multiple valves can be grown from a single cell population or a single patient explant of adequate length. FIG. 2 illustrates some examples of ways to create retrograde flow. FIG. 2A shows a channel 14 in cross section with a net fluid flow in the direction of arrows 16 and 18. By constricting the channel with “bumps” 20 and 22, higher magnitude retrograde flow can be produced at the apex of the bump and/or in the two regions 24 and 26 of the channel at appropriate pulsatile fluid flow rates. FIGS. 2B and 2C show a cross section of an initially open channel 28 that is subsequently constricted by expanding a pair of membranes 30 and 32 surrounding the channel. At appropriate pulsatile fluid flow rates, higher retrograde flow can be produced at the apex of bumps 20 and 22 and/or in regions 34 and 36. For example, a local increase in retrograde flow and negative shear force can be produced by forming a constriction in a blood vessel by injecting a small drop of a polymer or hydrogel near the vessel. The polymer or hydrogel drop pushes on the vessel wall to create a constriction in the lumen of the vessel, thus creating a retrograde fluid flow environment.

As will be apparent, the magnitude, flow rate, timing and other parameters of the retrograde flow for producing cardiac structures will depend in part on the particular scaffold and type of cells in the cell population. In various embodiments, retrograde flow can occur continuously or at intervals, and can increase or decrease in magnitude over time.

The cell population can include multipotent cells, pluripotent cells, totipotent cells, or any combination thereof. A multipotent cell (or multipotent progenitor cell) can give rise to cells from some but not all cell lineages. For example, a hematopoietic cell is a multipotent stem cell that can give rise to several types of blood cells, but not brain cells or other non-blood cells. A pluripotent cell can give rise to cells from any of the three germ layers—endoderm, mesoderm, ectoderm. A totipotent cell can give rise to cells of any type, including extra-embryonic tissues.

Embryonic stem cells are a type of pluripotent stem cell derived from the inner cell mass of blastocysts. The most common examples are mouse and human embryonic stem cells. Techniques for isolating and culturing embryonic stem cells have been developed (Thomson, J. A., et al., (1998) Science 282, 1145-1147; Evans, M. J., et al., (1981) Nature 292, 154-156; Hoffman, L. M., et al., (2005) Nat. Biotechnol. 23, 699-708). For example, mouse embryonic stem cells can be grown in medium supplemented with fetal calf serum in the presence of a feeder layer of inactivated mouse embryonic fibroblast cells. Mouse embryonic stem cells can also be grown in the absence of a feeder layer in culture medium that includes leukemia inhibitory factor. Human embryonic stem cells can be grown in the presence of a feeder layer of inactivated mouse embryonic fibroblast cells, or in the absence of a feeder layer on a substrate coated with a mouse tumor extract or other protein mixtures containing matrix proteins. Cultures of embryonic stem cells can be obtained from cells isolated from blastocysts, or from cells lines of mouse or human embryonic stem cells (Martin, G. R., (1981) Proc. Natl. Acad. Sci. U.S.A. 78, 7634-7638; Reubinoff, B. E., et al., (2000) Nat. Biotechnol. 18, 399-404).

Embryonic stem cells can be defined by the presence of certain transcription factors and cell surface markers. For example, mouse embryonic stem cells express transcription factor Oct4 and the cell surface protein SSEA-1, while human embryonic stem cells express transcription factor Oct4 and cell surface proteins SSEA3, SSEA4, Tra-1-60 and Tra-1-81.

Induced pluripotent stem cells are somatic cells that have been reprogrammed by forced expression of certain transcription factors including Oct4, Sox2 and Klf4. Methods for generating induced pluripotent stem cells include lentivirus or adenovirus delivery of relevant transcription factor genes into cells, transfection of plasmids containing relevant transcription factor gene into cells, and the use of valproic acid to increase reprogramming efficiency when various combinations of relevant transcription factor genes are introduced into cells (Takahashi, K., et al., (2006) Cell 126, 663-676; Stadtfeld, M., et al., (2008) Science 322, 945-949; Okita, K., et al., (2008) Science 322, 949-953; Huangfu, D., et al., (2009) Nat. Biotechnol. 26, 795-797). Induced pluripotent stem cells have the ability to differentiate into cells of all three germ layers.

Mesenchymal stem cells (MSCs) are a type of multipotent stem cell that can differentiate into vascular endothelial cell, bone cells, fat cells and cartilage cells. MSCs can be harvested from adult bone marrow and adipose tissue, or as freely circulating cells in the blood, then expanded and maintained in culture (Pittenge, M. F., et al, (2004) Circ. Res. 95. 9-20). For example, MSCs can be isolated from bone marrow cells by density gradient centrifugation in Ficoll followed by selection of adherent cells in culture or selection of MSCs by cell sorting.

The cell population can also include differentiated cells, which can be present alone or in combination with stem cells. Examples of differentiated cells include venous, arterial or valvular endothelial cells, epithelial cells, mesenchymal cells such as fibroblasts or myocytes, including cardiac muscle cells or vascular smooth muscle cells, or a combination thereof.

The kruppel-like factor (Klf) family of zinc finger transcription factors modulate cellular functions in a broad range of mammalian cell types and have important roles in cardiovascular biology. In cultured endothelial cells, the gene for Klf2 is activated by fluid shear stress. In vivo, Klf2 is associated with valve induction in the developing zebrafish heart. During development, Klf2 is normally expressed in valve precursors in response to pulsatile flow, and reducing its expression leads to a dysfunctional valve phenotype in the zebrafish (Hove, J. R., et al., (2003) Nature 421, 172-177; Vermot, J., et al., (2009) PLoS Biol 7(11): e1000246. doi:10.1371/journal.pbio.1000246). Thus, Klf2 appears to play a key role in linking hemodynamic forces to cardiovascular valve development.

In some embodiments, the expression of endogenous Klf2 in cells of the cell population is sufficient to support valve formation. In these cases, the shear stress produced during pulsatile flow is enough to stimulate Klf2 expression in the cell population. In some embodiments, however, Klf2 expression can be stimulated or increased to promote the formation of cardiovascular structures. For example, fluid flow can be modified to increase expression of the endogenous Klf2 gene. In particular embodiments, a steady shear force in the range of 0.001 to 100 dyn/cm² can be used as an epigenetic factor to induce the expression of Klf2. Another way of increasing Klf2 expression is to treat cells with compounds that induce Klf2 expression. For example, cells can be treated with statins, which are HMG-CoA reductase inhibitors that increase Klf2 expression (Parmar, K. M., et al., (2005) J. Biol. Chem. 280, 26714-26719).

In some embodiments, the amount of Klf2 can be increased by expression of an exogenous Klf2 gene inserted into cells of the cell population. For example, a Klf2 gene can be cloned into a viral vector. Following cell infection, the exogenous Klf2 gene can be expressed, leading to increased amounts of Klf2 gene product in the cell population. A number of virus expression systems are available, including those based on adenovirus, retrovirus, adeno-associated virus and herpesviruses (Stone, D., et al., (2000) J. Endocrinol. 164, 103-118). In another example, the Klf2 gene can be expressed from plasmids transfected into cells of the cell population, which can result in Klf2 expression without viral integration into the host cell genome.

Other genes can be expressed or activated to promote the formation of cardiovascular structures. Such genes include, but are not limited to: Notch genes, which encode transmembrane receptors involved in cell-to-cell interactions; and bone morphogenetic protein 1 (BMP-1) gene, which encodes a protease capable of inducing cartilage formation. Each gene acts through a well-defined pathway to achieve its biological effects.

Expression of the Klf2 gene can occur before, during or after applying the mechanical force, or any combination thereof. Similarly, expression of the Notch gene, the BMP-1 gene, or any combination of the Klf2, Notch and BMP-1 genes can occur before, during, after applying the mechanical force, or any combination thereof.

A scaffold is used to provide the cell population with support and a suitable growth environment. The scaffold is a structure that supports the growth and development of cells, and that allows for cell attachment and migration. In some embodiments, a scaffold can also potentially deliver biochemical and mechanical stimuli as well as nourishment to cells. Scaffolds can be prepared as individual components for use in bioreactors or formed as an integral part of a bioreactor, and can be fabricated from synthetic or naturally-occurring materials, or combinations of both.

For example, scaffolds can be prepared from polymers such as, but not limited to, poly(dimethylsiloxane) (PDMS), poly(glycerol sebacate) (PGS), biodegradable polymers such as polyglycolic acid (PGA), polylactic acid (PLA), lactic acid-glycolic acid copolymer (PLGA), poly-ε-caprolactone (PCL), polyamino acid, polyanhydride, and polyorthoester, or a combination thereof. In other embodiments, scaffolds can be prepared from hydrogels, which are cross-linked polymer networks that absorb water or other biological fluid. Examples of hydrogels include polyethyleneglycol (PEG), polyvinyl alcohol, polyvinyl pyrrolidone, polyethyleneimine, polyhydroxyethyl methacrylate family, polyacrylic acid, and polyacrylamide, or a combination thereof. Photopolymerizable hydrogels useful in tissue engineering include photopolymers having two or more reactive groups such as PEG acrylate derivatives, PEG methacrylate derivatives, polyvinyl alcohol derivatives, and modified polysaccharides such as hyaluronic acid or dextran methacrylate. Naturally-occurring hydrogels include carbohydrates such as hyaluronic acid, cellulose, or alginates, and proteins such as collagen or gelatin. Another type of hydrogel is an extracellular matrix secreted by Engelbreth-Holm-Swarm mouse sarcoma cells (commercially known as Matrigel, BD Biosciences, San Jose, Calif., USA). In particular embodiments, polymeric, fibrous or biodegradable scaffolds such as PGA mesh, PEG hydrogel, or a combination thereof are used.

Scaffolds can also be obtained from a biological source. For example, native tissue can be decellularized by treatment with detergents, leaving a scaffold of extracellular matrix. In some embodiments, a cardiovascular structure can be decellularized to provide a scaffold.

In some embodiments, proteins, peptides or hydrogels can be added to scaffolds to promote cell attachment, migration and/or growth. For example, collagen, fibrin or another extracellular matrix protein can be attached to polyethyleneglycol (PEG) to form a PEG-collagen scaffold (Dikovsky, D., et al., (2006) Biomaterials 27, 1496-506; Almany, L., et al., (2005) Biomaterials 26, 2467-2477), or in the case of human embryonic stem cells, a scaffold can be coated with a suitable hydrogel (for example, Matrigel), or with a cell feeder layer comprised of inactivated confluent mouse embryonic fibroblast cells.

A scaffold can be prepared by mixing cells with a polymeric material. For example, stem cells can be mixed with collagen-conjugated PEG precursor solution and the hydrogel formed by photopolymerization. The cell-laden PEG-collagen hydrogel can then be cultured in vitro or injected into a patient for in vivo tissue growth.

Scaffolds of various shapes can be prepared by techniques such as, but not limited to: molding, in which a hydrogel is formed in a suitably shaped mold; solvent casting and particulate leaching, in which a polymer is cast with pore-forming particles such as NaCl and then the particles are dissolved; electrospinning, in which continuous fibers are deposited on a substrate to form a porous network; emulsification and freeze drying, in which a polymer emulsion is freeze dried to form a porous scaffold. Microscale fabrication techniques include, but are not limited to: soft lithography, in which a polymeric stamp is prepared from patterned silicon wafers for printing or micromolding of scaffolds; and photolithography, in which patterns are formed on substrates using light (Khadmhosseini, A., et al., (2006) Proc. Natl. Acad. Sci. USA 103(8), 2480-2487). In some cases, a scaffold can be machined from a piece of teflon, PDMS, polyisoprene, poly(p-xylylene) polymers (also known as parylene), polyimide, titanium or any other solid biocompatible material. Scaffolds can also be prepared from photopolymerizable materials, allowing the formation of prescribed mechanical and chemical topology.

A scaffold can be prepared as a separate component of a bioreactor. In such cases, a bioreactor containing, for example, inlet and outlet ports for culture media flow can be prepared by combining the scaffold with components necessary to produce the inlet and outlet ports. In other cases, a scaffold is formed as an integral part of a bioreactor. In such cases, the bioreactor can be prepared, for example, using molding or a microscale fabrication technique similar to those used for scaffold preparation.

In some embodiments, one or more growth factors can be added to promote differentiation of cardiovascular structures. Examples of growth factors include, but are not limited to, vascular endothelial growth factor (VEGF), transforming growth factor beta (TGF-beta), insulin-like growth factor (IGF), bone morphogenetic protein (BMP), epidermal growth factor (EGF), fibroblast growth factor (FGF), platelet-derived growth factor (PDGF), or a combination thereof. The growth factor VEGF is a protein involved in blood vessel formation; TGF-beta is a protein involved in cellular differentiation and growth; IGF and PDGF are polypeptides that promote cell growth; EGF is a protein that stimulates the growth of epidermal and endothelial tissues; FGF is a protein that promotes endothelial cell growth.

In certain embodiments, the scaffold and cell population can be replaced with a patient-harvested explant of a blood vessel or other cardiovascular structure. The explant is then considered to include a scaffold and cell population. Retrograde flow can be applied to the explant by, for example, establishing a pulsatile flow with a retrograde component or by constricting the flow at a particular point in the blood vessel or cardiovascular structure. Following the formation of a valve or other cardiovascular structure in the explant, the explant or valve can be transplanted back into the same patient or into a different subject. In some embodiments, additional stem cells can be added to the explanted tissue to provide a cell population for cardiovascular structure formation. In other embodiments, the explant can be decellularized to provide a scaffold, then seeded with stem cells to produce a cell population. Retrograde flow is applied to the scaffold and cell population, leading to formation of a valve or other cardiovascular structure.

In another embodiment, the scaffold and cell population are injected into a subject in a region where valve formation is desired. The injection results in both shear forces and the proper environment for valve formation.

When a valve or other cardiovascular structure is produced for transplantation into an animal or human subject, the cells of the cell population can be autogeneic, allogeneic or xenogeneic, or a combination thereof. Similarly, when a valve or other cardiovascular structure is formed in an explant for transplantation, or when a scaffold and cell population is injected into a subject, the explant or the injected cell population can be autogeneic, allogeneic or xenogeneic, or a combination thereof.

In particular embodiments, a pulsatile flow bioreactor with a combination of hemodynamic forcing and a biochemical and mechanically tuned microenvironment is provided. The pulsatile flow bioreactor can be comprised of a 2D flow through a vessel or fluid conveying channel, or a 3D flow spanning two regions in separate planes, or a combination thereof. In some embodiments, the bioreactor is produced through microfabrication, micromachining or photogenerating methods using PDMS, another soft polymer, an extracellular matrix material, or a hydrogel. In some embodiments, geometry can be molded using a master mold such that scaffolds can be produced repeatedly. A bioreactor or flow cell employing either cellular expression of Klf2 or retrograde pulsatile flow, or a combination of both, for the development of tissue engineered valves or other cardiovascular structures is provided.

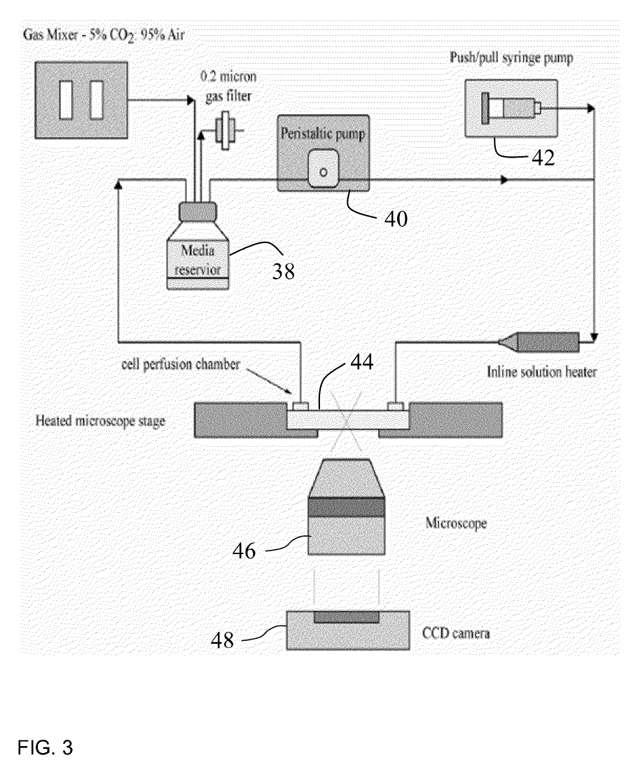

An example of a bioreactor system for growing a cardiovascular structure is shown in FIG. 3. The system includes a media reservoir 38 fluidly connected to a peristaltic pump 40. A push/pull syringe pump 42 is arranged to provide a pulsatile flow with a retrograde component to the system. The pumps are fluidly connected to a bioreactor 44, or cell perfusion chamber, which contains a scaffold and a cell population for cardiovascular structure formation. Growth and differentiation of the cell population can be monitored by viewing through a microscope 46, and images can be captured by a camera 48.

In further embodiments, the use of mechanical or hemodynamic forcing and Klf2 expression to differentiate and grow valves or other cardiovascular structures in a microfluidic architecture that functions as a component in micro total analysis systems (μTAS) or lab on chip (LOC) applications is provided.

According to various embodiments, a scaffold is seeded with stem cells, and the seeded cells are expanded to produce a cell population. During and/or after expansion, the shear profile of the cell culture is gradually changed to produce a retrograde shear force. Hemodynamic forcing is imposed by driving the culture medium under prescribed fluid mechanical conditions ultimately which involve inducing morphological changes to form bileaflet or trileaflet valves, or other cardiovascular structures, by imposing retrograde flow as well as the use of Klf2 as an intracellular factor.

The present invention may be better understood by referring to the accompanying examples, which are intended for illustration purposes only and should not in any sense be construed as limiting the scope of the invention.

### Example 1

Adult mesenchymal stem cell (MSCs) can be prepared from bone marrow samples by density gradient centrifugation in Ficoll (Ficoll-Paque, GE Healthcare Life Sciences, Piscataway, N.J., USA). A bone marrow sample is diluted with culture medium, then layered on the top of a Ficoll-Paque solution and centrifuged. Mononuclear cells are seeded, then MSCs is purified by adherence to plastic.

### Example 2

A cell culture bioreactor with a geometry capable of producing the desired shear conditions is produced through microfabrication, micromachining or photogeneration.

After seeding the bioreactor, MSCs are given approximately 6 hours to attach before starting the flow regiment. The cells are exposed to a steady shear at forces less than or equal to 1 dyn/cm² to align colony morphology to the flow. After 1 day under steady flow, the media is switched to include VEGF and TGF-Beta, and the shear profile is changed to a pulsatile flow with a retrograde component, exposing the cell culture layer to retrograde shear forces where the retrograde time span increases relative to forward time span from 0 to 50% over a period of a week. MSCs are then maintained in culture under the final flow conditions for a period of 1 to 2 months.

Cells are seeded at a density of approximately 5000-6000 cells/cm² and cultured in MSCGM™ Mesenchymal Stem Cell Growth Medium (Lonza, Walkersville, Md., USA), or in low glucose DMEM or M-199 medium with 10% Fetal Bovine Serum and 100 U/ml pen/strep, at 37° C. and 5% CO₂. Media is changed every 3-4 days throughout the process.

Valve formation is identified by the beginning of out-of-plane growth and the eventual functioning of the formed cardiovascular structure as a tissue capable of rectifying or blocking media flow. The tissue construct can be removed from the bioreactor for implantation into a subject, which can be an animal or human.

### Example 3

A hydrogel scaffold formed of methylcellulose, alginate, PEG-fibrinogen, or Matrigel is injected into a subject in a region where valve formation is desired. The injection creates both the biochemical environment to promote valve growth as well as the shear forces to promote formation of bileaflet valves.

### REFERENCES

The following publications are incorporated by reference herein in their entirety.
Almany, L., et al., (2005) Biomaterials 26, 2467-2477.
Dikovsky, D., et al., (2006) Biomaterials 27, 1496-506.
Evans, M. J., et al., (1981) Nature 292, 154-156.
Hoffman, L. M., et al., (2005) Nat. Biotechnol. 23, 699-708.
Hove, J. R., et al., (2003) Nature 421, 172-177.
Huangfu, D., et al., (2009) Nat. Biotechnol. 26, 795-797.
Khademhosseini, A., et al., (2006) Proc. Natl. Acad. Sci. USA 103(8), 2480-2487.
Martin, G. R., (1981) Proc. Natl. Acad. Sci. U.S.A. 78, 7634-7638.
Okita, K., et al., (2008) Science 322, 949-953.
Parmar, K. M., a al, (2005) 1 Biol. Chem. 280, 26714-26719).
Reubinoff, B. E., et al., (2000) Nat. Biotechnol. 18, 399-404.
Stadtfeld, M., et al., (2008) Science 322, 945-949.
Stone, D., et al., (2000) J. Endocrinol. 164, 103-118.
Takahashi, K., et al., (2006) Cell 126, 663-676.
Thomson, J. A., et al., (1998) Science 282, 1145-1147.
Vermot, J., et al., (2009) PLoS Biol 7(11): e1000246. doi:10.1371/journal.pbio.1000246.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of forming a cardiovascular structure in culture, comprising
applying mechanical force to a cell population in culture such that a cardiovascular structure is formed.

2. The method of claim 1, wherein the mechanical force comprises a shear force.

3. The method of claim 1, wherein the mechanical force results from pulsatile retrograde fluid flow.

4. The method of claim 1, wherein the mechanical force is transferred through a fluid or cell culture medium.

5. The method of claim 1, wherein the cell population comprises multipotent, pluripotent or totipotent cells, or cardiovascular cells, or a combination thereof.

6. The method of claim 1, wherein the cell population is supported by a scaffold prepared from an explanted cardiovascular structure.

7. The method of claim 1, wherein the mechanical force is produced by forming a constriction that creates a local increase in the magnitude of retrograde shear force on the cell population.

8. The method of claim 1, wherein the cardiovascular structure is a blood vessel or a valve.

9. The method of claim 1, further comprising increasing expression of an endogenous or inserted Klf2 gene, Notch gene, BMP gene, or a combination thereof, in the cell population wherein the increasing expression occurs before, during or after applying the mechanical force, or a combination of thereof.

10. A method of forming a cardiovascular valve in culture, comprising:
applying retrograde fluid flow to a cell population in culture such that a cardiovascular valve is formed.

11. The method of claim 10, wherein the retrograde fluid flow is a pulsatile retrograde fluid flow.

12. The method of claim 10, wherein the retrograde fluid flow is applied through a cell culture medium.

13. The method of claim 10, wherein the cell population comprises multipotent, pluripotent or totipotent cells, or cardiovascular cells, or a combination thereof.

14. The method of claim 10, wherein the cell population is supported by a scaffold prepared from an explanted vein or artery.

15. The method of claim 10, further comprising increasing expression of an endogenous or inserted Klf2 gene, Notch gene, or BMP gene, or a combination thereof, in the cell population wherein the increasing expression occurs before, during or after applying the retrograde fluid flow, or a combination of thereof.

16. A culture vessel that applies a mechanical force to a cell population, comprising:
a scaffold for supporting a cell population; and
means for producing a pulsatile fluid flow in the culture vessel;
wherein the scaffold comprises at least one section in which retrograde fluid flow results from movement of the pulsatile fluid flow through the scaffold.

17. The culture vessel of claim 16, wherein the scaffold is a synthetically prepared support or a component of a cardiovascular system, or a combination thereof.

18. The culture vessel of claim 16, wherein the culture vessel is a bioreactor or a flow cell.

19. A tissue engineered cardiovascular structure prepared according to claim 1.

20. A tissue engineered cardiovascular valve prepared according to claim 10.
